(19) United States
(12) Patent Application Publication
(10) Pub. No.: US 2010/0093817 A1
(43) Pub. Date: Apr. 15, 2010

(54) Compounds

(75) Inventors: Patrício Manuel Vieira Araújo SOARES DA SILVA, Porto (PT); David Alexander LEARMONTH, Alfena (PT); Alexander BELIAEV, Mindelo (PT)

Correspondence Address: CONLEY ROSE, P.C., 5601 GRANITE PARKWAY, SUITE 750, PLANO, TX, 75024, US

(73) Assignee: BIAL - PORTELA & CA S.A., S. Mamede do Coronado (PT)

(21) Appl. No.: 12/524,940

(22) PCT Filed: Jan. 31, 2008
(86) PCT No.: PCT/PT2008/000004

§ 371 Date: Oct. 13, 2009

(30) Foreign Application Priority Data
Feb. 1, 2007 (GB) 0701968.0
Feb. 1, 2007 (GB) 0701969.8

(51) Int. Cl. C07D 405/02; A61K 31/4178

(52) U.S. Cl. 514/389; 548/311.4

(57) ABSTRACT

This invention relates to new 6,8-difluorochroman-3-yl-1,3-dihydroimidazole-2-thiones, their preparation, and their use as a medicament.

### CROSS-REFERENCE TO RELATED APPLICATIONS

This application is a filing under 35 U.S.C. 371 of International Application No. PCT/PT2008/000004 filed Jan. 31, 2008, entitled “Compounds,” claiming priority of Great Britain Application Nos. 0701968.0 and 0701969.8, both filed on Feb. 1, 2007, which applications are incorporated by reference herein in their entirety.

### FIELD OF THE INVENTION

This invention relates to peripherally-selective inhibitors of dopamine-β-hydroxylase (NH), their method of their preparation, and their use as a medicament.

### BACKGROUND OF THE INVENTION

In recent years, interest in the development of inhibitors of DβH has centred on the hypothesis that inhibition of this enzyme may provide significant clinical improvements in patients suffering from cardiovascular disorders such as hypertension or chronic heart failure. The rationale for the use of DβH inhibitors is based on their capacity to inhibit the biosynthesis of noradrenaline, which is achieved via enzymatic hydroxylation of dopamine. Activation of neurohumoral systems, chiefly the sympathetic nervous system, is the principal clinical manifestation of congestive heart failure (Parmley, W. W., Clinical Cardiology, 18: 440-445, 1995). Congestive heart failure patients have elevated concentrations of plasma noradrenaline (Levine, T. B. et al., Am. J. Cardiol., 49:1659-1666, 1982), increased central sympathetic outflow (Leimbach, W. N. et al., Circulation, 73: 913-919, 1986) and augmented cardiorenal noradrenaline spillover (Hasking, G. J. et al., Circulation, 73:615-621, 1966). Prolonged and excessive exposure of myocardium to noradrenaline may lead to down-regulation of cardiac β₁-adrenoceptors, remodelling of the left ventricle, arrhythmias and necrosis, all of which can diminish the functional integrity of the heart. Congestive heart failure patients who have high plasma concentrations of noradrenaline also have the most unfavourable long-term prognosis (Cohn, J. N. et al., N. Engl. J. Med., 311:819-823, 1984). Of greater significance is the observation that plasma noradrenaline concentrations are already elevated in asymptomatic patients with no overt heart failure and can predict ensuing mortality and morbidity (Benedict, C. R. et al., Circulation, 94:690-697, 1996). This implies that the activated sympathetic drive is not merely a clinical marker of congestive heart failure, but may contribute to progressive worsening of the disease.

Inhibition of sympathetic nerve function with adrenoceptor antagonists appeared a promising approach, however a significant proportion of patients do not tolerate the immediate haemodynamic deterioration that accompanies β-blocker treatment (Pfeffer, M. A. et al., N. Engl. J. Med., 334:1396-7, 1996). An alternative strategy for directly modulating sympathetic nerve function is to reduce the biosynthesis of noradrenaline via inhibition of DβH, the enzyme responsible for conversion of dopamine to noradrenaline in sympathetic nerves. This approach has several merits including gradual modulation as opposed to abrupt inhibition of the sympathetic system, and causing increased release of dopamine, which can improve renal function such as renal vasodilation, diuresis and natriuresis. Therefore inhibitors of DβH may provide significant advantages over conventional β-blockers.

Several inhibitors of DβH have been thus far reported in the literature. Early first and second generation examples such as disulfiram (Goldstein, M. et al., Life Sci., 3:763, 1964) and diethyldithiocarbamate (Lippmann, W. et al., Biochem. Pharmacol., 18: 2507, 1969) or fusaric acid (Hidaka, H. Nature, 231, 1971) and aromatic or alkyl thioureas (Johnson, G. A. et al, J. Pharmacol. Exp. Ther., 171: 80, 1970) were found to be of low potency, exhibited poor selectivity for DβH and caused toxic side effects. The third generation of DβH inhibitors however, were found to have much greater potency, such as for example, nepicastat (RS-25560-197, IC₅₀ 9 nM) (Stanley, W. C., et al., Br. J Pharmacol., 121: 1803-1809, 1997), which was developed to early clinical trials. Although devoid of some of the problems associated with first and second generation DβH inhibitors, a very important discovery was that nepicastat was found to cross the blood brain barrier (BBB), thereby able to cause central as well as peripheral effects, a situation which could lead to undesired and potentially serious CNS side-effects of the drug. Therefore there yet remains an unfulfilled clinical requirement for a potent, non-toxic and peripherally selective inhibitor of DβH, which could be used for treatment of certain cardiovascular disorders. A DβH inhibitor with similar or even greater potency than nepicastat, but devoid of CNS effects (inability to cross the BBB) would provide a significant improvement over all DβH inhibitor compounds thus far described in the prior art.

Dopamine-β-hydroxylase inhibitors are also disclosed in WO95/29165. Furthermore, WO 2004/033447 discloses dopamine-β-hydroxylase inhibitors having high potency and significantly reduced brain access, giving rise to potent and peripherally selective DβH inhibitors. WO2004/033447, discloses that (R)-5-(2-aminoethyl)-1-(6,8-difluorochroman-3-yl)-1,3-dihydroimidazole-2-thione and its pharmaceutically acceptable salts, in particular the hydrochloride salt, are especially advantageous DβH inhibitors. This compound has the structure shown in formula I:

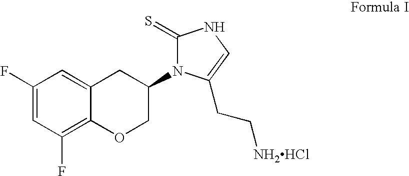

### SUMMARY OF THE INVENTION

We have now found useful compounds that are metabolites of the compound of formula I.

In accordance with one aspect of the invention there is provided a compound of formula II:

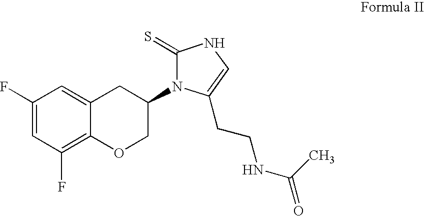

The compound of formula II can be prepared by acetylation of the free base 18 of the compound of formula I with acetic anhydride and triethylamine in a solvent, which preferably comprises a mixture of methanol and dichloromethane:

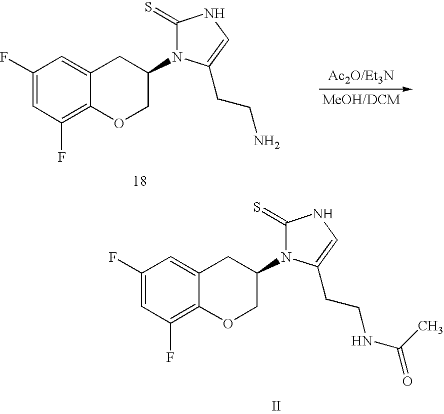

In accordance with another aspect of the invention there is provided a compound of formula III:

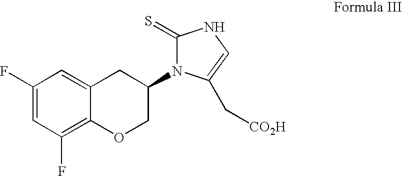

The compound of formula III can be formed by cyclocondensation of the aminochroman 2 with hydroxy ketone 3 (Meul et al., 1987, Chimia 41(3) pp. 73-76) and a water soluble thiocyanate, especially an alkali metal thiocyanate, such as potassium thiocyanate, in the presence of an organic acid, especially AcOH, which acts as a reagent, and can also serve the function of providing a solvent for the reaction (a separate solvent could be provided, if desired) followed by the alkaline hydrolysis of the intermediate ester 4:

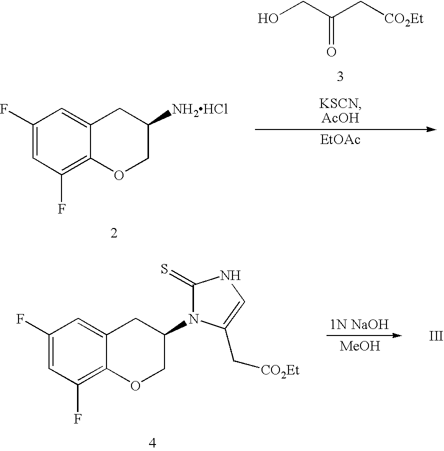

Preparation of hydroxyl ketone 3 is described in Meul et al., 1987, Chimia 41(3) pp. 73-76.

Compound 2 may be synthesised starting from L-serine methyl ester hydrochloride by condensation of its N-trityl derivative with 2,4-difluorophenol under Mitsunobu conditions followed by deprotection, ethoxycarbonylation of the resulting amino acid, Friedel-Crafts cyclization of N-protected derivative and reduction of the ethoxycarbonylamino ketone. The alkaline hydrolysis of ethyl carbamate gives 2:

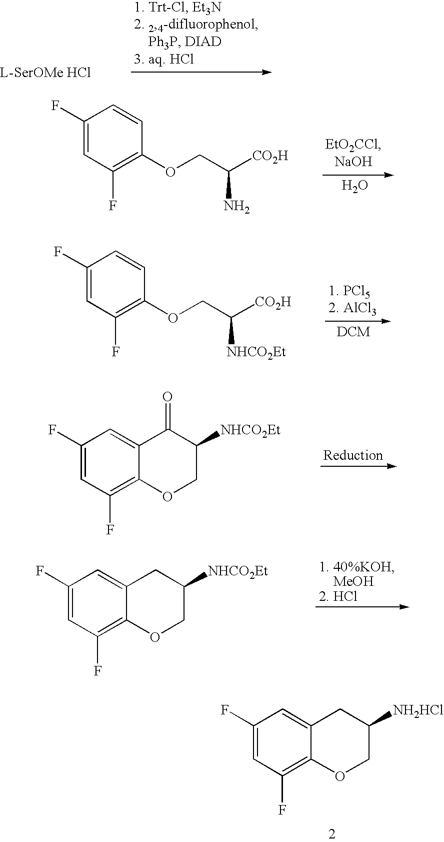

In accordance with another aspect of the invention there is provided a compound of formula IV:

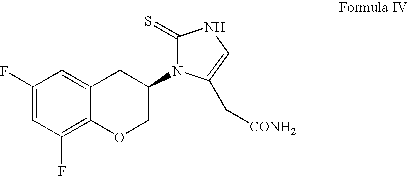

The compound of formula IV can be formed by the azide activation of the compound of formula III and reaction of the intermediate acyl azide (not shown) with ethanolic ammonia:

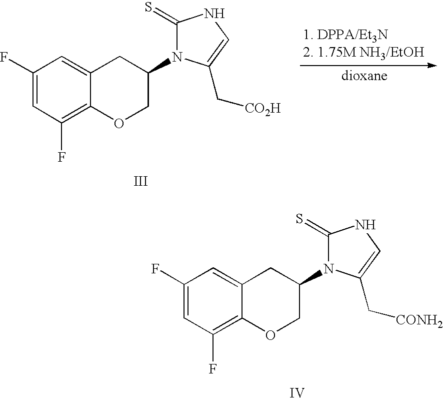

In accordance with another aspect of the invention there is provided a compound of formula V:

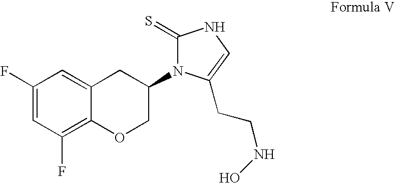

To prepare building block 11, which is necessary for the synthesis of the compound of formula V, commercially available racemic 1,2,4-butantriol (5) was protected as the isopropylidene derivative 6 and converted to the tosylate 7 (Börjesson et al., 1992 Tetrahedron 48(30) pp. 6325-6334), which was then used for the alkylation of N,O-di-Boc-hydroxylamine (Carpino et al., 1959 J. Am. Chem. Soc. 81 pp. 955-957). The isopropylidene protection of the fully substituted hydroxylamine 8 was removed with p-TsOH in methanol without affecting the Boc groups. The primary OH group of the diol 9 was selectively silylated followed by oxidation of the secondary alcohol 10 to give the target intermediate 11. Cyclocondensation of the aminochroman 2 with compound 11 and a water soluble thiocyanate, especially an alkali metal thiocyanate, such as potassium thiocyanate, in the presence of an organic acid, especially AcOH, which acts as a reagent, and can also serve the function of providing a solvent for the reaction (a separate solvent could be provided, if desired) afforded N,O-di-Boc protected derivative 12 which, upon deprotection with a mixture of 2N HCl and formic acid in dioxane, yielded the compound of formula V:

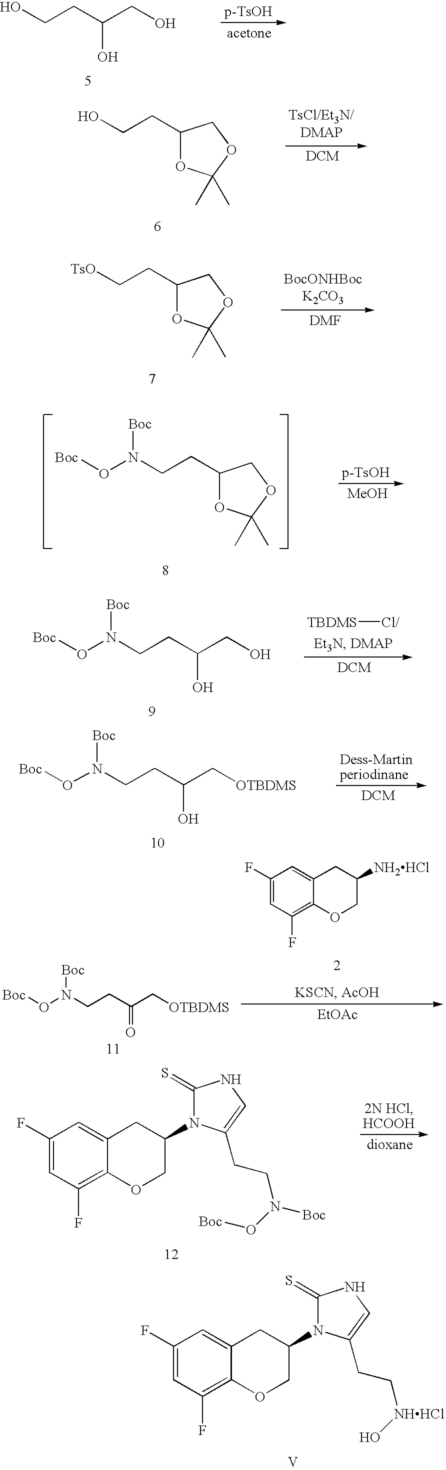

In accordance with one aspect of the invention there is provided a compound of formula VI:

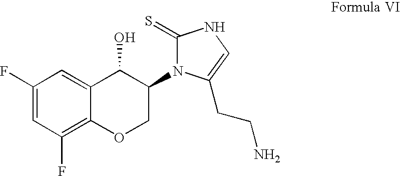

The compound of formula VI may be provided as the free base, or as a pharmaceutically acceptable salt thereof. For example, the hydrochloride salt of the compound of formula VI can be prepared by the process similar to one described in WO2004/033447 which consists of the cyclocondensation of aminochromanol 6′ with protected hydroxy ketone 7′ (preparation of 7′ is given in WO2004/033447) followed by a deprotection and a ring opening of the intermediate 8′. Aminochromanol 6′ may be synthesised starting from L-serine methyl ester hydrochloride (1′) by condensation of its N-trityl derivative with 2,4-difluorophenol under Mitsunobu conditions followed by deprotection, trifluoroacetylation of the resulting amino acid (2′), Friedel-Crafts cyclization of N-protected derivative (3′) and reduction of the trifluoroacetylamino ketone (4′). The acid hydrolysis of trifluoroacetamide (5′) gives 6′:

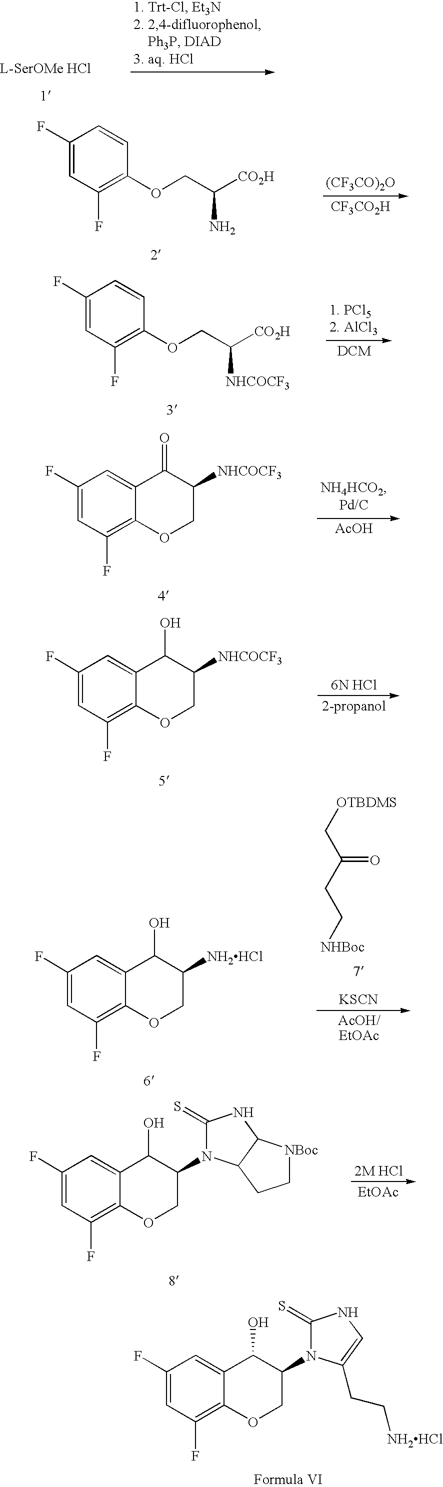

In accordance with another aspect of the invention there is provided a compound of formula VII:

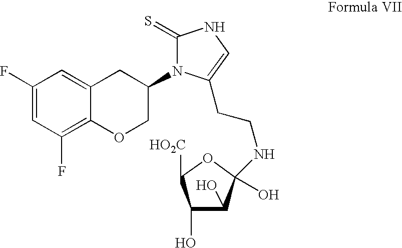

The compound of formula VII can be prepared by reacting of the free base 18 with D-glucuronic acid in methanol at 60° C. for 1 hour:

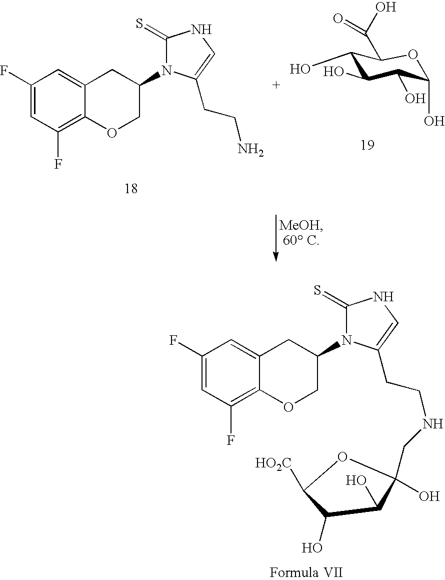

In accordance with another aspect of the invention there is provided a compound of formula VIII:

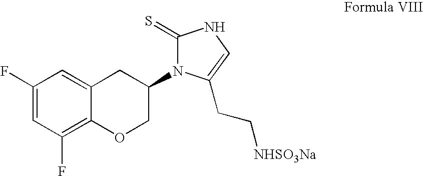

The compound of formula VIII can be prepared by treatment of the free base 18 with SO₃-trimethylamine complex followed by the cation exchange with the sodium form of Amberlyst XN 1010:

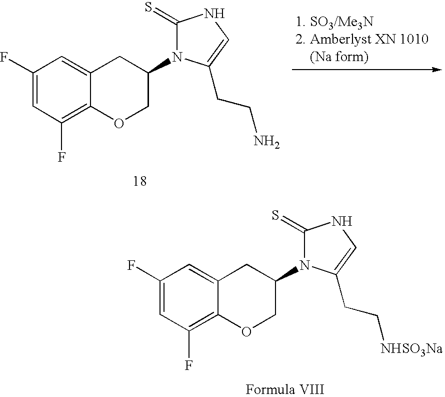

In accordance with another aspect of the invention there is provided a compound of formula IX:

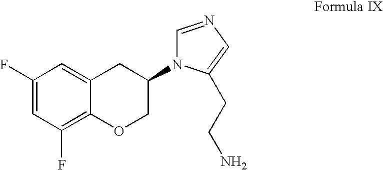

The compound of formula IX may be provided as the free base, or as a pharmaceutically acceptable salt thereof. For example, the hydrochloride salt of the compound of formula IX can be prepared by oxidative desulfurisation of the phthalyl derivative 20 with peracetic acid followed by deprotection of the imidazole 21:

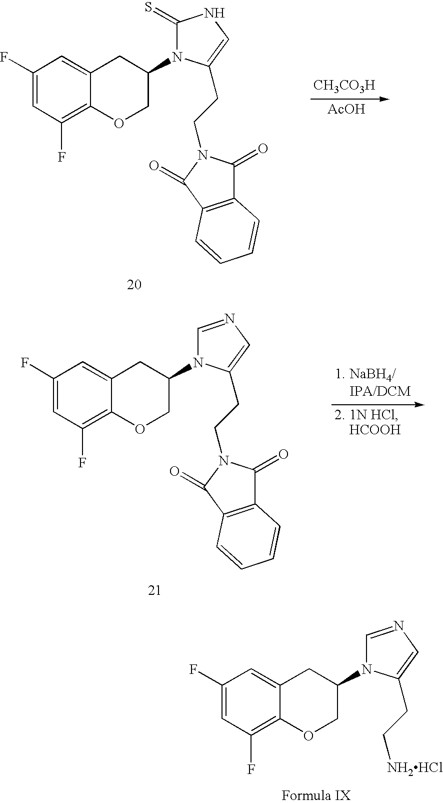

The compound of formula 20 may be made by reacting a compound of formula 22:

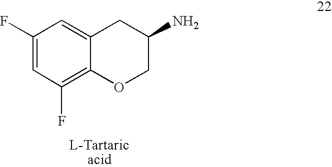

with a compound of formula 23:

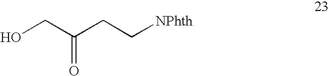

and a water soluble thiocyanate, especially an alkali metal thiocyanate, such as potassium thiocyanate, and acetic acid.

Compound 22 may be synthesised starting from L-serine methyl ester hydrochloride by condensation of its N-trityl derivative with 2,4-difluorophenol under Mitsunobu conditions followed by deprotection, ethoxycarbonylation of the resulting amino acid, Friedel-Crafts cyclization of N-protected derivative and reduction of the ethoxycarbonylamino ketone. The alkaline hydrolysis of ethyl carbamate gives 22:

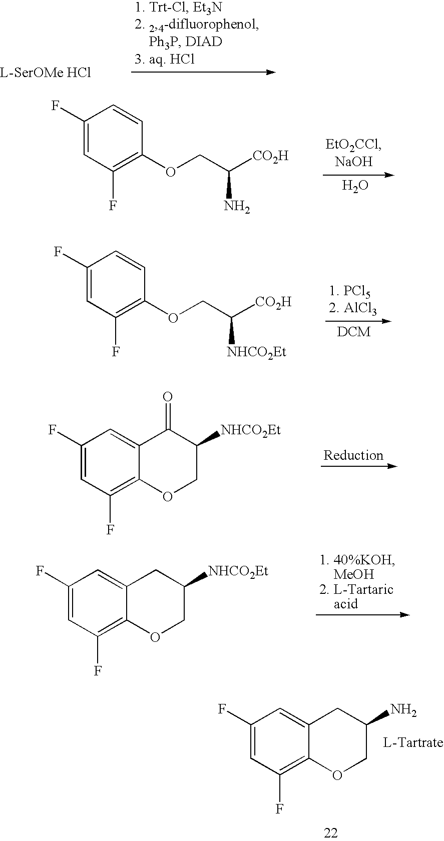

### DETAILED DESCRIPTION

According to another aspect of the invention there is provided a compound of formula II, III, IV, V, VI, VII, VIII or IX in isolated form.

The compounds of formula II, III, IV, V, VI, VII, VIII or IX may be provided in the form of the free base, or in the form of pharmaceutically acceptable salts, such as the hydrochloride or sodium salt. Esters of the compounds of formula II, III, IV, V, VI, VII, VIII or IX are also encompassed by the application.

According to another aspect of the invention there is provided a pharmaceutical composition comprising a therapeutically effective amount of a compound of formula II, III, IV, V, VI, VII, VIII or IX in combination with a pharmaceutically effective carrier.

For the preparation of pharmaceutical compositions of compounds of formula II, III, IV, V, VI, VII, VIII or IX, inert pharmaceutically acceptable carriers are admixed with the active compounds. The pharmaceutically acceptable carriers may be either solid or liquid. Solid form preparations include powders, tablets, dispersible granules and capsules. A solid carrier can be one or more substances which may also act as diluents, flavouring agents, solubilizers, lubricants, suspending agents, binders or tablet disintegrating agents; it may also be an encapsulating material.

Preferably the pharmaceutical preparation is in unit dosage form, e.g. packaged preparation, the package containing discrete quantities of preparation such as packeted tablets, capsules and powders in vials or ampoules.

The dosages may be varied depending on the requirement of the patient, the severity of the disease and the particular compound being employed. For convenience, the total daily dosage may be divided and administered in portions throughout the day. It is expected that once or twice per day administration will be most suitable. Determination of the proper dosage for a particular situation is within the skill of those in the medical art.

According to another aspect of the invention there is provided a compound of formula II, III, IV, V, VI, VII, VIII or IX as described above, for use as a medicament.

According to another aspect of the invention there is provided the use of a compound of formula II, III, IV, V, VI, VII, VIII or IX as described above, in the manufacture of a medicament for treating disorders where a reduction in the hydroxylation of dopamine to noradrenaline is of therapeutic benefit.

As used herein, the term treatment and variations such as ‘treat’ or ‘treating’ refer to any regime that can benefit a human or non-human animal. The treatment may be in respect of an existing condition or may be prophylactic (preventative treatment). Treatment may include curative, alleviation or prophylactic effects.

According to another aspect of the invention there is provided the use of a compound of formula II, III, IV, V, VI, VII, VIII or IX as described above, in the manufacture of a medicament for treating a subject afflicted by one or more anxiety disorders.

According to another aspect of the invention there is provided the use of a compound of formula II, III, IV, V, VI, VII, VIII or IX as described above, in the manufacture of a medicament for treating migraine.

According to another aspect of the invention there is provided the use of a compound of formula II, III, IV, V, VI, VII, VIII or IX as described above, in the manufacture of a medicament for treating a subject afflicted by one or more cardiovascular disorders.

According to another aspect of the invention there is provided the use of a compound of formula II, III, IV, V, VI, VII, VIII or IX as described above, in the manufacture of a medicament for treating hypertension, chronic heart failure, congestive heart failure, angina, arrythmias or circulatory disorders such as Raynaud's Phenomenon.

According to another aspect of the invention there is provided the use of a compound of formula II, III, IV, V, VI, VII, VIII or IX as described above, in the manufacture of a medicament for use in inhibiting dopamine-β-hydroxylase.

According to another aspect of the invention there is provided a method of treating migraine comprising administering a therapeutically effective amount of a compound of formula II, III, IV, V, VI, VII, VIII or IX as described above to a patient in need thereof.

According to another aspect of the invention there is provided a method of treating anxiety disorders comprising administering a therapeutically effective amount of a compound of formula II, III, IV, V, VI, VII, VIII or IX as described above to a patient in need thereof.

According to another aspect of the invention there is provided a method of treating cardiovascular disorders comprising administering a therapeutically effective amount of a compound of formula II, III, IV, V, VI, VII, VIII or IX as described above to a patient in need thereof.

According to another aspect of the invention there is provided a method of treating hypertension comprising administering a therapeutically effective amount of a compound of formula II, III, IV, V, VI, VII, VIII or IX as described above to a patient in need thereof.

According to another aspect of the invention there is provided a method of treating chronic heart failure comprising administering a therapeutically effective amount of a compound of formula II, III, IV, V, VI, VII, VIII or IX as described above to a patient in need thereof.

According to another aspect of the invention there is provided a method of treating congestive heart failure comprising administering a therapeutically effective amount of a compound of formula II, III, IV, V, VI, VII, VIII or IX as described above to a patient in need thereof.

According to another aspect of the invention there is provided a method of treating angina comprising administering a therapeutically effective amount of a compound of formula II, III, IV, V, VI, VII, VIII or IX as described above to a patient in need thereof.

According to another aspect of the invention there is provided a method of treating arrythmias comprising administering a therapeutically effective amount of a compound of formula II, III, IV, V, VI, VII, VIII or IX as described above to a patient in need thereof.

According to another aspect of the invention there is provided a method of treating circulatory disorders such as Raynaud' s Phenomenon comprising administering a therapeutically effective amount of a compound of formula II, III, IV, V, VI, VII, VIII or IX as described above to a patient in need thereof.

### Experimental Section

Dopamine-β-hydroxylase activity was evaluated by the ability to β-hydroxylate dopamine to noradrenaline as previously described (Kojima, K., Parvez, S. and Nagatsu T. 1993. Analysis of enzymes in catecholamine biosynthesis. In Methods in Neurotransmitter and Neuropeptide Research, pp. 349-380: Elsevier Science Publishers). SK-N-SH cells (ATCC HTB-11), a human neuroblastoma derived cell line, were used as a source of human dopamine-β-hydroxylase. SK-N-SH cells cultured in 24 well plates were preincubated for 20 min in a reaction medium containing 200 mM sodium acetate, 30 mM N-ethylmaleimide, 5 μM copper sulphate, 0.5 mg/ml catalase aqueous solution, 1 mM pargyline, 10 mM sodium fumarate and 20 mM ascorbic acid. Thereafter, cells were incubated for further 45 min in the reaction medium with added increasing concentrations of dopamine (0.5 to 100 mM). During preincubation and incubation, the cells were continuously shaken and maintained at 37° C. The reaction was terminated by the addition of 0.2 M perchloric acid. The acidified samples were stored at 4° C. before injection into the high pressure liquid chromatograph for the assay of noradrenaline. In experiments conducted with the aim of studying the effects of new dopamine-β-hydroxylase inhibitors on enzyme activity, test compounds (0.3 to 10,000 nM) of interest were added to the preincubation and incubation solutions; the incubation was performed in the presence of a concentration (50 mM) of dopamine 2.5 times the corresponding Kₘ value as determined in saturation experiments.

Summary table on the profile of compound I metabolites as dopamine-β-hydroxylase (DβH) inhibitors under in vitro experimental conditions

| | | In vitro DβH |
|---|---|---|
| | In vitro DβH | inhibition |
| | inhibition | (IC₅₀ μM) |
| Compound I | + | 0.2 |
| compound VI | + | NE |
| compound VII | + | NE |
| compound VIII | + | NE |
| compound V | + | 0.5 |
| compound IV | + | 1.8 |
| compound IX | − | NE |
| compound III | + | 0.6 |
| compound II | + | 0.5 |

NE = not evaluated;
+ = inhibitory activity

Effects of Compound VIII on Human DβH In Vitro Assay

| | % control |
|---|---|
| Compound | Mean ± SEM |
| Vehicle | 100.0 ± 4.2 |
| Nepicastat (5 μM) | 0.4 ± 0.2 |
| compound VIII (0.5 μM) | 32.1 ± 1.1 |
| compound VIII (1 μM) | 20.3 ± 0.7 |
| compound VIII (5 μM) | 3.5 ± 1.1 |

### EXAMPLES

The invention disclosed herein is exemplified by the following examples of preparation, which should not be construed to limit the scope of the disclosure. Alternative pathways and analogous structures may be apparent to those skilled in the art.

### Example 1

### (R)-N-(2-(3-(6,8-difluorochroman-3-yl)-2-thioxo-2,3-dihydro-1H-imidazol-4-yl)ethyl)acetamide (II)

To a solution of 1 (0.15 g, 0.5 mmol) in a mixture of dichloromethane (3 ml) and methanol (1 ml), triethylamine (0.084 ml, 0.6 mmol) was added in one portion at 20-25° C. followed by acetic anhydride (0.057 ml, 0.6 mmol) in one portion at 20-25° C. The mixture was stirred for 30 min and then evaporated to dryness under reduced pressure. The residue was partitioned between dichloromethane (5 ml) and 0.5N HCl (5 ml). The organic phase was separated and dried (MgSO₄) and then evaporated to approx. 1 ml. The mixture was diluted with petroleum ether (3 ml), the solid was collected, washed with petroleum ether and dried in air. Yield was 0.14 g (80%) with the product exhibiting no clear melting point (softens at 120° C.).

### Example 2

### Ethyl (R)-2-(3-(6,8-difluorochroman-3-yl)-2-thioxo-2,3-dihydro-1H-imidazol-4-yl)acetate (4)

Aminochroman 2 (0.2 g, 0.9 mmol), hydroxy ketone 3 (0.15 g, 1 mmol) and potassium thiocyanate (0.097 g, 1 mmol) were heated under reflux with stirring for 6 h under nitrogen in a mixture of ethyl acetate (2 ml) and acetic acid (0.2 ml). After cooling to 20-25° C. the mixture was diluted with petroleum ether (2 ml) and washed with NaHCO₃ solution. The organic layer was dried (MgSO₄) and evaporated under reduced pressure. The residue was purified on a silica gel column using mixtures of ethyl acetate and petroleum ether (1:2 to 1:1 v/v) as eluent. Fractions containing the product were collected and evaporated under reduced pressure to give a viscous oil, yield 0.24 g (75%).

### Example 3

### (R)-2-(3-(6,8-difluorochroman-3-yl)-2-thioxo-2,3-dihydro-1H-imidazol-4-yl)acetic acid (III)

To a solution of the ester 4 (0.32 g, 0.9 mmol) in methanol (5 ml) was added. 1N NaOH in water (1.3 ml, 1.3 mmol) was added in one portion at 20-25° C. The mixture was stirred for 1 h and then evaporated to dryness under reduced pressure. The residue was taken up into water (5 ml) and insoluble material was filtered off. The filtrate was acidified with 2N HCl to pH 1-2 and cooled in ice for 1 h. The precipitate was collected, washed with cold water, then dried under vacuum at 20-25° C. Yield was 0.21 g (72%) with the product exhibiting decomposition without melting.

### Example 4

### (R)-2-(3-(6,8-difluorochroman-3-yl)-2-thioxo-2,3-dihydro-1H-imidazol-4-yl)acetamide (IV)

To a suspension of the acid III (0.20 g, 0.613 mmol) in dioxane (5 ml) triethylamine (0.15 ml, 1.08 mmol) was added at 20-25° C. in one portion to give a clear solution. The solution was cooled to 15° C. DPPA (0.15 ml, 0.674 mmol) was added dropwise and the mixture was stirred at 15-20° C. for 1 h. A 1.75 M solution of ammonia in ethanol (3.5 ml, 6.13 mmol) was added dropwise at 15° C. and the mixture was stirred for 1 h. The precipitate was filtered off and the filtrate was evaporated to dryness. The residue was distributed between EtOAc-petroleum ether (1:1) mixture and water, the precipitate in both phases was collected, washed with water, EtOAc-petroleum ether (1:1) mixture and dried in vacuum at 50° C. Yield was 0.092 g (46%), and the product had mp. 253° C. (dec.).

### Example 5

### tert-Butyl tert-butoxycarbonyloxy(3,4-dihydroxybutyl)carbamate (9)

N,O-Di-Boc hydroxylamine (2.11 g, 9.06 mmol), tosylate 7 (3.22 g, 10.72 mmol) and finely ground potassium carbonate (1.85 g, 13.4 mmol) were stirred at 20-25° C. for 16 h in DMF (10 ml). The mixture was distributed between EtOAc-petroleum ether (1:1) mixture (50 ml) and brine (50 ml), the organic phase was separated and washed with brine, dried (MgSO₄) and evaporated under reduced pressure. The residue was taken up into MeOH (40 ml). p-TsOH monohydrate (1.90 g, 10 mmol) was added in one portion at 20-25° C. and the mixture was stirred for 1 h. The solution was neutralised with triethylamine (1.4 ml, 10 mmol) and evaporated to dryness. The residue was applied on a column and eluted with a EtOAc-petroleum ether (1:1) mixture. Fractions containing product were collected and evaporated under reduced pressure to give an oil, in a yield of 2.2 g (76%).

### Example 6

### tert-Butyl tert-butoxycarbonyloxy(4-(tert-butyldimethylsilyloxy)-3-hydroxybutyl)carbamate (10)

A mixture of the diol 9 (2.2 g, 6.85 mmol), TBDMS-Cl (1.19 g, 7.88 mmol), triethylamine (1.1 ml, 7.88 mmol) and DMAP (0.010 g, 0.8 mmol) were stirred in anhydrous DCM (60 ml) for 24 h at 20-25° C. The solution was washed with water (50 ml), dried (MgSO₄) and evaporated under reduced pressure. The residue was purified on a column using petroleum ether and a petroleum ether-ethyl acetate (4:1) mixture as eluents. Fractions containing product were collected and evaporated under reduced pressure to give oil in a yield of 2.6 g (87%).

### Example 7

### tert-Butyl tert-butoxycarbonyloxy(4-(tert-butyldimethylsilyloxy)-3-oxobutyl)carbamate (11).

To a solution compound 10 (5.05 g, 11.59 mmol) in dry DCM (90 ml) Dess-Martin periodinane (4.91 g, 11.59 mmol) was added in one portion at 20-25° C. The mixture was stirred for 1 h, concentrated under reduced pressure to approx. 20 ml, applied on a column and eluted with petroleum ether and a petroleum ether-ethyl acetate (9:1) mixture. Fractions containing product were collected and evaporated under reduced pressure to give an oil in a yield of 4.83 g (96%).

### Example 8

### tert-Butyl (R)-tert-butoxycarbonyloxy(2-(3-(6,8-difluorochroman-3-yl)-2-thioxo-2,3-dihydro-1H-imidazol-4-yl)ethyl)carbamate (12)

A mixture of aminochroman 2 (1.01 g, 4.54 mmol), compound 11 (1.97 g, 4.54 mmol), potassium thiocyanate (0.45 g, 4.68 mmol), acetic acid (1.8 ml) and water (0.082 ml) was heated in ethyl acetate (18 ml) under reflux with stirring for 7 h under nitrogen. After cooling to 20-25° C. the mixture was diluted with petroleum ether (18 ml), washed with NaHCO₃ solution, organic layer was dried (MgSO₄) and evaporated under reduced pressure. The residue was separated on a silica gel column using mixtures of ethyl acetate and petroleum ether (1:2 to 2:1 v/v) as eluent. Fractions containing the product were collected and evaporated under reduced pressure to give a solid foam. The yield was 1.06 g (44%).

### Example 9

### (R)-1-(6,8-difluorochroman-3-yl)-5-(2-(hydroxyamino)ethyl)-1H-imidazole-2(3H)-thione hydrochloride (V)

N,O-di-Boc derivative 12 (0.114 g, 0.216 mmol) was stirred in the mixture of 2N HCl in water (0.5 ml, 1 mmol) and formic acid (0.2 ml) in dioxane (2 ml) at 80° C. for 0.5 h. The brown solution was evaporated to dryness under vacuum, the residue was taken up into 2-propanol (2 ml) and diluted with ether (4 ml). The solid was filtered off and the filtrate was diluted with petroleum ether (6 ml). The solid was collected, washed with petroleum ether, dried in vacuum at room temperature. Yield was 0.038 g (49%) and the product exhibited decomposition without melting.

### Example 10

### (R)-5-(2-Aminoethyl)-1-(6,8-difluorochroman-3-yl)-1,3-dihydroimidazole-2-thione. 18

(R)-5-(2-Aminoethyl)-1-(6,8-difluorochroman-3-yl)-1,3-dihydroimidazole-2-thione hydrochloride (9.64 g, 27.72 mmol) was dissolved in water (160 ml) at 40-45° C. with stirring. To the resulting solution 2-propanol (64 ml) was added, the mixture was cooled to 35-38° C., dichloromethane (256 ml) was added followed by 1N NaOH (28 ml, 28 mmol) and the stirring continued for 10-15 min. Lower organic phase was separated, dried over MgSO₄ and evaporated under reduced pressure to approx. 40 ml. The resulting suspension was diluted with petroleum ether (200 ml), the precipitate was collected, was with petroleum ether on the filter, dried in vacuum. Yield 7.8 g (91%), mp 192-5° C. (dec).

### Example 11

### (R)-2-Amino-3-(2,4-difluorophenoxy)propionic acid (2′)

The vessel was purged with nitrogen followed by a charge of L-serine methyl ester hydrochloride (1′) (25 kg) and dichloromethane (400 kg, 300 L). The temperature of the vessel contents were maintained using glycol cooling (temp range 15-25° C.). Triethylamine (33.4 kg) was charged to the vessel over 45 min. A solution of trityl chloride (45.7 kg) in dichloromethane (265 kg) was prepared and charged to the vessel over 3 hours maintaining the temperature between 15-25° C. The resultant reaction mixture was stirred for 6 hours at 25-30° C. HPLC analysis confirmed complete reaction.

Water (263 kg) was charged to the vessel and the mixture stirred for 30 minutes and allowed to settle 30 minutes. The lower organic phase was separated off and then the top aqueous phase was extracted with DCM (90 kg). The combined organic phase was recharged to the vessel (after removal of aqueous phase) and toluene (450 kg) was added. The DCM was distilled out using vacuum distillation (base temperature less than 35° C. and atmospheric pressure used initially followed by application of vacuum down to 200 mbar). The distillate was monitored for DCM/toluene content by GC.

The contents of the vessel were then cooled to below 30° C. using cooling water and the reactor was vented with nitrogen. 2,4-Difluorophenol (21.3 kg) was charged to the vessel followed by triphenylphoshine (42.4 kg). The mixture was stirred out for 30 min. Maintaining the reaction temperature in the range 25-30° C., the diisopropyl azodicarboxylate (DIAD, 40.9 kg) was charged over 3 hours and 30 min. The reaction mixture was stirred for a further 4 hours before sampling. The reaction was analysed by HPLC and showed no starting material.

6N Hydrochloric acid (400 kg) was charged to the reaction mixture in the vessel and the mixture was warmed gently to reflux (observed temp=79.3° C.). The mixture was held at reflux for a further 4 hours before being cooled to 60-65° C. The mixture was allowed to settle for 1 hour at 60-65° C. before separating off the lower aqueous phase. The organic was extracted with 2N hydrochloric acid (20 kg) at 60-65° C. and the aqueous was combined with the first aqueous phase.

The combined aqueous phase was cooled to 20-30° C. and then the pH was adjusted to pH 6.8-7.2 using 32% w/w sodium hydroxide solution (294.5 kg used). The resultant suspension was stirred for 1 hour and the pH was checked and adjusted as necessary. The solids were filtered off and the filter cake was washed with water (175 L). The solid on the filter was then re-slurried with acetone (140 kg) and filtered. The solid was pulled down as dry as possible (21.3 kg damp weight) and then dried at 40-45° C./100-60 mbar. Yield=15.25 kg (as dry product)

### Example 12

### (R)-N-(6,8-difluoro-4-oxochroman-3-yl)-2,2,2-trifluoroacetamide (4′)

The amino acid 2′ (4.34 g, 20 mmol) was dissolved in TFA (18 ml) at room temperature with stiffing during 20-25 min. The solution was cooled in the ice-bath and TFAA (4.22 ml, 30 mmol) was added dropwise. The mixture was stirred in the ice-bath for 2 hours, ice (ca. 10 g) was added. The mixture was allowed to warm up to the room temperature and evaporated in vacuo. The residue was dissolved in dichloromethane (100 mL), the solution was washed with water, brine, dried over MgSO₄ and evaporated to dryness. The resulting viscous oil (crude compound 3′, 6.25 g) was dissolved in anhydrous DCM (25 mL) and added dropwise to a suspension of PCl₅ (4.45 g, 21.25 mmol) in anhydrous DCM (25 mL) with the ice cooling. The resulting solution was stirred for 1 hour in the ice bath and added dropwise to suspension of AlCl₃ (8.66 g, 65 mmol) in anhydrous DCM (50 mL). The mixture was stirred for 2.5 hours at room temperature, refluxed for 1 hour, cooled, poured on a mixture of ice (ca. 100 g) and conc. HCl (10 mL), diluted with DCM (150 mL) and allowed to warm up to room temperature with stirring. The organic phase was washed with water, NaHCO₃ solution, brine, dried over MgSO₄ and evaporated in vacuo until the beginning of crystallisation. The mixture was diluted with the equal volume of petroleum ether and placed in the fridge for crystallization for 3-4 hours. The crystals were filtered, washed with petroleum ether. Yield 4.87 g (82.5%), mp 167-8° C.

### Example 13

### (3S)-3-Amino-6,8-difluorochroman-4-ol hydrochloride (6′)

Protected amino ketone 4′ (1.48 g, 5 mmol) was heated at 80° C. with stirring in acetic acid (20 ml) with 5% Pd/C (0.5 g) and ammonium formate (0.63 g, 20 mmol) for 2 h, then another portion of 5% Pd/C (0.5 g) and ammonium formate (0.63 g, 20 mmol) was added and stirring continued for 1 h. The catalyst was filtered off on a Celite layer, the filtrate was evaporated to dryness. The residue was taken up into EtOAc (50 ml), the solution was washed with water, dried (MgSO₄), evaporated to dryness under reduced pressure, the residue was dissolved in toluene (50 ml) and evaporated to dryness under reduced pressure to give crude 5′ (1.52 g) as an oil. The oil was heated with stirring under reflux in a mixture of 2-propanol (12 ml) and 6N HCl (12 ml) for 1 h, evaporated to dryness under reduced pressure, the residue was dissolved in 2-propanol (15 ml) and evaporated to dryness under reduced pressure. The solid residue was re-slurried in a mixture of ethyl acetate and petroleum ether (2:1 v/v), the crystals were collected washed with the mixture of ethyl acetate and petroleum ether (2:1 v/v), dried in vacuum. Yield 1.01 g (83%), decomposes without melting.

### Example 14

### 5-(2-Aminoethyl)-1-((3S ,4S)-6,8-difluoro-4-hydroxychroman-3-yl)-1H-imidazole-2(3H)-thione hydrochloride (Formula VI)

The mixture of aminochromanol 6′ (0.35 g, 1.47 mmol), compound 7′ (0.61 g, 1.91 mmol), potassium thiocyanate (0.19 g, 1.91 mmol), acetic acid (0.6 ml) and water (0.035 ml) was heated in ethyl acetate (6 ml) under reflux with stiffing for 8 h under nitrogen. After cooling to 20-25° C. the mixture was diluted with petroleum ether (18 ml), washed with NaHCO₃ solution, organic layer was dried (MgSO₄) and evaporated under reduced pressure. The residue was separated on a silica gel column using mixtures of petroleum ether and ethyl acetate (3:1 v/v) as eluent. Fractions containing the product were collected and evaporated under reduced pressure to give 0.257 g of solid material. This was stirred in 1M HCl in EtOAc (6 ml) for 4 h at 20-25° C., the precipitate was collected, washed with EtOAc and dried in vacuum to give 0.18 g of solid, not homogenous by TLC. To purify was stirred in the mixture of water (1.5 ml) and 10% 2-propanol in DCM with addition of 3N NaOH to pH 9-10. The aqueous phase was separated, acidified to pH 1 with 3N HCl and evaporated to dryness under reduced pressure. The solid residue was taken up into absolute ethanol (10 ml), inorganic salts were filtered off, the filtrate was evaporated to dryness under reduced pressure, the residue was re-precipitated with ether from 2-propanol. Yield 0.038 g (7%), decomposes without melting.

### Example 15

### 5-((2-(3-((R)-6,8-Difluorochroman-3-yl)-2-thioxo-2,3-dihydro-1H-imidazol-4-yl)ethylamino)methyl)-3,4,5-trihydroxytetrahydrofuran-2-carboxylic acid (Formula VII)

The amine 18 (0.31 g, 1 mmol) and D-glucuronic acid (0.2 g, 1.03 mmol) in methanol (10 ml) were stirred at 60° C. under nitrogen for 1 h. After cooling to 20-25° C. dark insoluble material was filtered off, the filtrate was diluted with ether (40 ml), the precipitate was collected, washed with ether, dried. Yield 0.32 g (65%), decomposes without melting.

### Example 16

### Sodium (R)-2-(3-(6,8-difluorochroman-3-yl)-2-thioxo-2,3-dihydro-1H-imidazol-4-yl)ethylsulfamate (Formula VIII)

Compound 18 (0.15 g, 0.5 mmol) and SO₃.Me₃N complex (0.07 g, 0.5 mmol) were stirred in MeOH (5 ml) at 20-25° C. for 2 h to produce a clear solution. To the solution MeONa (0.027 g, 0.5 mmol) was added, the solution was filtered to remove solid particles and evaporated to dryness under reduced pressure. The solid residue was re-dissolved in MeOH (3 ml) and precipitated with ether (up to 10 ml). The solid was collected, washed with ether, 2-propanol, dried to give 0.13 g of product, by NMR still contained 30% of the Me₃N salt. Re-dissolved in MeOH (2 ml), shaken with Amberlyst XN 1010 (1 g, Na form, washed with water and methanol) for 1 h at 20-25° C., the resin was filtered off. The filtrate was evaporated to dryness, the residue was triturated in ether to produce 0.06 g (29%) of solid, decomposes without melting.

### Example 17

### (R)-2-(2-(1-(6,8-Difluorochroman-3-yl)-1H-imidazol-5-yl)ethyl)isoindoline-1,3-dione (21)

To a suspension of compound 20 (0.44 g, 1 mmol) in AcOH (5 ml) 39% solution of peracetic acid in AcOH (0.85 ml, 5 mmol) was added at 20-25° C. A clear solution formed which was stirred at 20-25° C. for 30 min and added drop wise to aqueous sodium bicarbonate solution. The resulting mixture was extracted with dichloromethane (25 ml), washed with sodium bicarbonate solution, brine and evaporated to dryness under reduced pressure to give a solid foam, yield 0.37 g (90%).

### Example 18

### (R)-2-(1-(6,8-Difluorochroman-3-yl)-1H-imidazol-5-yl)ethanamine (Formula IX)

To a suspension of 21 (0.35 g, 0.86 mmol) in the mixture of 2-propanol (7.5 ml), water (1.3 ml) and DCM (0.94 ml) NaBH₄ (0.16 g, 4.2 mmol) was added at 20° C. in one portion with stiffing. The mixture was stirred at 20° C. for 16 h, 1N HCl (1.3 ml) and formic acid (0.65 ml) were added dropwise. DCM was distilled off until head temperature reached 76-78° C., the mixture was stirred under reflux for 1.5 h and cooled to room temperature. Water (10 mL) was added, 2-propanol was removed on a rotavap, the residue was washed with EtOAc-petroleum ether (2:1 v/v) mixture (2×10 mL). To the aqueous layer 10% 2-propanol in DCM solution (20 mL) was added with stiffing followed by 5N NaOH to pH 9-10. Organic layer was separated, dried (MgSO₄), evaporated to dryness. The residue was dissolved in 2-propanol (10 mL) and 3M HCl in abs. EtOH (1 mL) was added (pH of the mixture ca. 1). The mixture was diluted with ether (20 ml) and stirred at 5° C. for 2 h, the crystals were collected, washed with ether, dried in vacuum at 40° C. Yield 0.21 g (78%).

It will be appreciated that the invention described above may be modified within the scope of the claims.

1. A compound having the following formula, or a pharmaceutically acceptable salt or ester thereof, in isolated form: 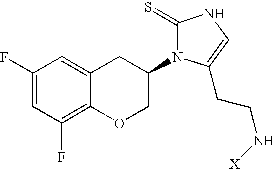
wherein X is —OH or —SO₃Na.

2-9. (canceled)

10. The compound according to claim 1, wherein the pharmaceutically acceptable salt is the hydrochloride salt.

11. A pharmaceutical composition comprising a therapeutically effective amount of a compound according to claim 1, in combination with a pharmaceutically effective carrier.

12. The compound according to claim 1, wherein the compound is formulated for use as a medicament.

13. A method comprising utilizing the compound according to claim 1, in the manufacture of a medicament for treating disorders where a reduction in the hydroxylation of dopamine to noradrenaline is of therapeutic benefit.

14. A method comprising utilizing the use of a compound according to claim 1, in the manufacture of a medicament for treating a subject afflicted by a condition selected from the group consisting of one or more anxiety disorders; migraine; cardiovascular disorders; hypertension, chronic heart failure, congestive heart failure, angina, arrythmias, circulatory disorders, and Raynaud's Phenomenon.

15-17. (canceled)

18. A method comprising utilizing the compound according to claim 1, in the manufacture of a medicament for use in inhibiting dopamine-β-hydroxylase.

19. A method of treating a condition selected from the group consisting of one or more anxiety disorders; migraine; cardiovascular disorders; hypertension, chronic heart failure, congestive heart failure, angina, arrythmias, circulatory disorders, and Raynaud's Phenomenon, comprising administering a therapeutically effective amount of a compound according to claim 1 to a patient in need thereof.

20-33. (canceled)

34. A method of preparing a compound of formula V 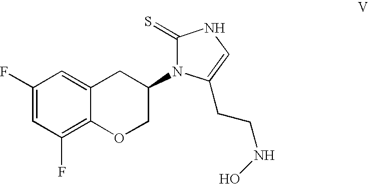
comprising the following steps:
(a) reaction of 1,2,4-butantriol to form a compound of formula 6 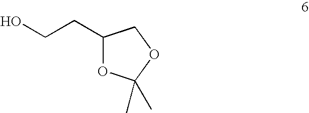
(b) conversion of the compound of formula 6 to a compound of formula 7: 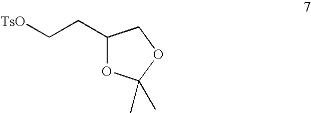
(c) alkylation of N,O-di-Boc-hydroxylamine with the compound of formula 7;
(d) removal of the isopropylidene protection from the product of step (c);
(e) silylation of the product of step (d) followed by oxidation to form a compound of formula 11: 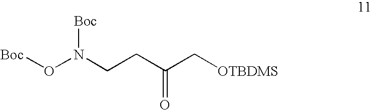
(f) cyclo condensation of a compound of formula 2 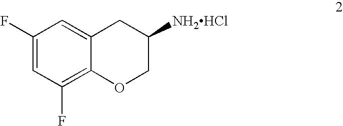
with a compound of formula 11 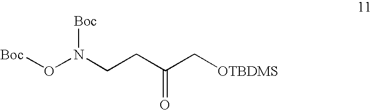
and a water soluble thiocyanate to form a compound of formula 12; and 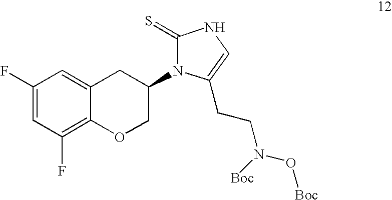
(g) removal of the Boc protecting groups from the compound of formula 12.

35. The method according to claim 34, wherein step (f) is performed in the presence of an organic acid.

36. The method according to claim 34, wherein the water soluble thiocyanate is an alkali metal thiocyanate, preferably potassium thiocyanate.

37. A method of producing the hydrochloride salt of the compound of formula V 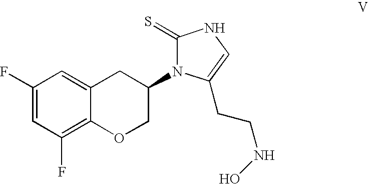
comprising producing the free base of formula V using a process according to claim 34, wherein step (g) is performed using a mixture of HCl and formic acid in dioxane.

38-40. (canceled)

41. A method of preparing a compound of formula VIII: 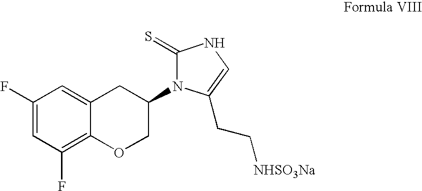
comprising treatment of a compound of formula 18: 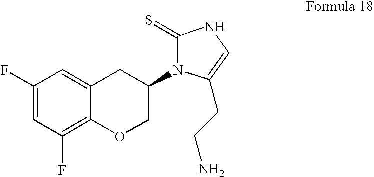
with a SO₃⁻ complex followed by a cation exchange reaction.

42. The method according to claim 41, wherein the SO₃⁻ complex is a SO₃⁻ trimethylamine complex.

43. The method according to claim 41, wherein the cation exchange reaction is carried out with the sodium form of amberlyst XN 1010.

44-45. (canceled)

46. The method according to claim 35, wherein the water soluble thiocyanate is an alkali metal thiocyanate, preferably potassium thiocyanate.

47. The method of producing the hydrochloride salt of the compound of formula V 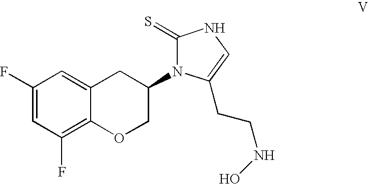
comprising producing the free base of formula V using a process according to claim 35, wherein step (g) is performed using a mixture of HCl and formic acid in dioxane.

48. The method according to claim 42, wherein the cation exchange reaction is carried out with the sodium form of amberlyst XN 1010.

49. A compound of formula V, or a pharmaceutically acceptable salt or ester thereof:

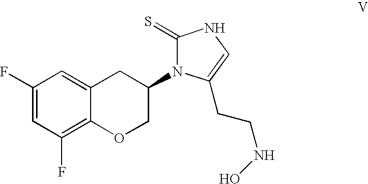

50. A compound of formula VIII, or a pharmaceutically acceptable salt or ester thereof: [chemical structure]
